(19) United States
(12) Patent Application Publication
(10) Pub. No.: US 2024/0066154 A1
(43) Pub. Date: Feb. 29, 2024

(54) IRON OXIDE MAGNETIC PARTICLES

(71) Applicant: ZTI BIOSCIENCES CO., LTD., Daejeon (KR)

(72) Inventors: Hyungseok CHANG, Suwon-si (KR); Yong-Sun PARK, Seoul (KR); Ji-Young RYU, Yongin-si (KR)

(73) Assignee: ZTI BIOSCIENCES CO., LTD., Daejeon (KR)

(21) Appl. No.: 18/270,118

(22) PCT Filed: Dec. 28, 2021
(86) PCT No.: PCT/KR2021/020044

§ 371 Date: Jun. 28, 2023

(30) Foreign Application Priority Data
Dec. 29, 2020 (KR) 10-2020-0185966
Nov. 18, 2021 (KR) 10-2021-0159440

(51) Int. Cl. A61K 49/18; C01G 49/02

(52) U.S. Cl. CPC A61K 49/1818; C01G 49/02; B82Y 15/00

(57) ABSTRACT

The present invention provides iron oxide magnetic particles containing iron oxide and MXₙ, wherein M as a transition metal containing electrons in a 5d orbital on the periodic table includes one or more selected from the group consisting of Hf, Ta, W, Re, Os, Ir, Pt, Au, and Hg, X includes one or more selected from the group consisting of F, Cl, Br, and I, and n is an integer of 1 to 6.

### CROSS REFERENCE TO RELATED APPLICATIONS

This application is a National Stage of International Application No. PCT/KR2021/020044 filed Dec. 28, 2021, claiming priority based on Korean Patent Application No. 10-2020-018566 filed Dec. 29, 2020 and 10-2021-0159440 filed Nov. 18, 2021, the entire disclosures of which are incorporated herein by reference.

### TECHNICAL FIELD

The present invention relates to iron oxide magnetic particles.

### BACKGROUND ART

Magnetic particles have been widely used in biomedical fields including cell labeling, magnetic resonance imaging (MRI), drug delivery, and thermotherapy. Superparamagnetic iron oxide magnetic particles among various types of magnetic particles have been widely studied in the field of biomedicine because of their high magnetic susceptibility and superparamagnetism.

In addition, since magnetic particles have a characteristic of generating heat when radiation or a magnetic field is applied thereto, they can also be used in contrast agents for magnetic resonance imaging (MRI), magnetic carriers for drug delivery in the field of nanomedicine, magnetic or radiation-based thermotherapy, and the like.

In the field of imaging diagnosis, iron oxide is a superparamagnetic contrast agent, and is proposed as a negative contrast agent. However, iron oxide particles have a strong hydrophobic attraction so that they may be well agglomerated with each other to form clusters or rapidly biodegrade when exposed to a biological environment. In addition, if the structure of iron oxide particles is not sufficiently stable, the original structure may be changed, and thus the magnetic properties thereof may be changed, and the iron oxide particles may have toxicity. Meanwhile, iodine is proposed as a positive contrast agent, but a formulation technology that increases the content per volume of the contrast medium has also been introduced due to a problem that liver/kidney toxicity occurs when it is used at high concentrations in order to enhance the contrast effect.

Meanwhile, in order to supplement the limitations of existing cancer treatment methods, thermotherapy based on radiation or electromagnetic fields has been proposed (Wust et al. Lancet Oncology, 2002, 3:487-497). One of the unique characteristics of cancer cells is that their ability to adapt to heat is significantly inferior to that of normal cells. Thermotherapy is an anticancer therapy that selectively annihilates cancer cells by raising the temperature of the cancer tissue and its surroundings to about 40 to 43° C. by using the difference in thermal sensitivity between normal cells and cancer cells in this way. When injecting magnetic particles around cancer cells to apply a magnetic field from the outside, heat may be generated from the magnetic particles to annihilate the cancer cells in a short time. Since the magnetic field is not affected by skin tissue so that there is no limit to the penetration depth, heat may be selectively applied when magnetic particles are accumulated in cancer tissue in the body. Therefore, research on thermotherapy using magnetic particles has received a lot of attention.

Iron oxide magnetic particles are also mainly used as magnetic particles for thermotherapy. This is because iron oxide magnetic particles are materials having an indirect band gap in which energy equal to the amount of momentum used is converted into heat and released. Among them, Fe₃O₄ (magnetite) or a-Fe (ferrite)-based magnetic particles have biocompatibility, heat induction ability, chemical stability, and unique magnetic properties. Because of these characteristics, research as a self-heating element for thermotherapy of iron oxide magnetic particles is currently being actively conducted, and has been approved for medical use by the US FDA. However, Fe₃O₄ particles among iron oxide magnetic particles are nano-sized, and their crystalline phase easily changes to α-Fe₂O₃, γ-Fe₂O₃, etc. depending on the conditions of the surrounding environment, and accordingly, the heating characteristics and their magnetic properties change, and thus there is a disadvantage of reducing the ability to generate heat. Research on Co, Ni, and Mg-based MFe₂O₄ (M=Co, Ni, and Mg) particles as another materials is being conducted, but this also has a disadvantage in that it is difficult to apply it to the inside of a living body due to a low exothermic temperature.

### PRIOR ART DOCUMENT

### Non-Patent Document

Wust et al. Lancet Oncology, 2002, 3:487-497.

### DISCLOSURE

### Technical Problem

A problem to be solved by the present invention is to provide iron oxide magnetic particles that can be used in various fields.

### Technical Solution

In order to solve the above-described problem, the present invention provides iron oxide magnetic particles containing iron oxide and MXₙ, wherein M as a transition metal containing electrons in a 5d orbital on the periodic table includes one or more selected from the group consisting of Hf, Ta, W, Re, Os, Ir, Pt, Au, and Hg, X includes one or more selected from the group consisting of F, Cl, Br, and I, and n is an integer of 1 to 6.

### Advantageous Effects

The iron oxide magnetic particles of the present invention may have high reactivity to stimuli introduced from outside such as radiation, magnetic fields, and radio waves.

In addition, the contrast agent containing the iron oxide magnetic particles is used and can be applied to various diagnostic imaging devices, and can be administered in a small amount to obtain sufficient images.

The iron oxide magnetic particles of the present invention can have high structural stability due to a bond formed between iron oxide and a halogen compound and a transition metal element containing electrons in a 5d orbital.

### DESCRIPTION OF DRAWINGS

FIG. 1 is transmission electron microscope (TEM) photographs of iron oxide magnetic particles of Example 3.

FIG. 2 is results of XPS component analysis of the iron oxide magnetic particles of Example 3.

### MODES OF THE INVENTION

In one embodiment, at least a portion of the surface of the iron oxide magnetic particles may be additionally coated with a hydrophilic polymer to form a complex. The hydrophilic polymer may be introduced to increase solubility in water and stabilization of the iron oxide magnetic particles according to one embodiment, or to enhance targeting or penetration force into specific cells such as cancer cells. Such a hydrophilic polymer may preferably have biocompatibility, and may include, for example, one or more selected from the group consisting of polyethylene glycol, polyethylene amine, polyethyleneimine, polyacrylic acid, polymaleic anhydride, polyvinyl alcohol, polyvinylpyrrolidone, polyvinyl amine, polyacrylamide, polyethylene glycol, phosphoric acid-polyethylene glycol, polybutylene terephthalate, polylactic acid, polytrimethylene carbonate, polydioxanone, polypropylene oxide, polyhydroxyethyl methacrylate, starch, dextran derivatives, amino sulfonic acids, sulfonic acid peptides, silica, and polypeptides, but is not limited thereto. If necessary, in the case of targeting cancer cells, peptides or proteins containing folic acid, transferrin, or RGD may be used as the hydrophilic polymer, and hyaluronidase or collagenase may be used to enhance penetration force to cells, but are not limited thereto.

In one embodiment, the iron oxide may be derived from complexes of iron and one or more compounds selected from the group consisting of an aliphatic carboxylic acid salt having 4 to 25 carbon atoms and an amine-based compound. Examples of the aliphatic carboxylic acid salt having 4 to 25 carbon atoms may include one or more selected from the group consisting of butyrate, valerate, caproate, enanthate, caprylate, pelargonate, caprate, laurate, myristate, pentadecylate, acetate, palmitate, palmitoleate, margarate, stearate, oleate, vaccenate, linoleate, (9,12,15)-linolenate, (6,9,12)-linolenate, eleostearate, tuberculostearate, arachidate, arachidonate, behenate, lignocerate, nervonate, cerotate, montanate, melissate, and a peptide salt containing one or more amino acids. These compounds may be used alone or in the form of a mixed acid salt of two or more thereof.

The metal component of the aliphatic carboxylic acid salt having 4 to 25 carbon atoms may include one or more selected from the group consisting of calcium, sodium, potassium and magnesium.

Examples of the amine-based compound may include one or more selected from the group consisting of methylamine, ethylamine, propylamine, isopropylamine, butylamine, amylamine, hexylamine, octylamine, 2-ethylhexylamine, nonylamine, decylamine, laury lamine, pentadecylamine, cetylamine, stearylamine, cyclohexylamine, dimethylamine, diethylamine, dipropylamine, diisopropylamine, dibutylamine, diamylamine, dioctylamine, di(2-ethylhexyl)amine, didecylamine, dilaurylamine, dicetylamine, distearylamine, methylstearylamine, ethylstearylamine, butylstearylamine, triethylamine, triamylamine, trihexylamine, trioctylamine, triallylamine, oleylamine, laurylaniline, stearylaniline, triphenylamine, N,N-dimethylaniline, dimethylbenzylaniline, monoethanolamine, diethanolamine, triethanolamine, dimethylaminoethanol, diethylenetriamine, triethylenetetramine, tetraethylenepentamine, benzylamine, diethylaminopropylamine, xylylenediamine, ethylenediamine, hexamethylenediamine, dodecamethylenediamine, dimethylethylenediamine, triethylenediamine, guanidine, diphenylguanidine, N, N, N′, N′-tetramethyl-1,3-butanediamine, N,N,N′,N′-tetramethylethylenediamine, 2,4,6-tris(dimethylaminomethyl)phenol, morpholine, N-methylmorpholine, 2-ethyl-4-methylimidazole, and 1,8-diazabicyclo (5,4,0) undecene-7 (DBU).

The iron oxide magnetic particles of the present invention may be prepared by adjusting the mol % of MXₙ to about 1 to 13 mol % of a complex of iron and one or more compounds selected from the group consisting of aliphatic carboxylic acids and aliphatic amines.

In one embodiment, the iron oxide magnetic particles may contain MXₙ at a weight ratio of 1:0.005 to 0.08, preferably 1:0.01 to 0.08, based on iron oxide. The ratio may be measured using inductively coupled plasma mass spectroscopy, which is metal content analysis equipment. When MXₙ is contained within the above-described range in the iron oxide magnetic particles of the present invention, excellent specific loss power may be secured, and high temperature change may be secured under an external alternating magnetic field or when radiation rays are irradiated.

The iron oxide magnetic particles as described above can secure high specific loss power while having high reactivity to stimuli introduced from the outside, such as radiation, magnetic fields, and radio waves, so that they can be effectively used for thermotherapy described later.

Presumably, a compound such as MXₙ of the present invention not only may increase the intensity of magnetization by combining with iron oxide, which is a magnetic material, but also increase the size or total amount of electromagnetic field energy that the compound can absorb, thereby enabling the amount of thermal energy emitted from the final iron oxide-based magnetic particles to be increased. This may improve or increase high thermal energy emission (conversion) efficiency (ILP: Intrinsic loss power) compared to existing iron oxide-based magnetic particles, even in the electromagnetic field energy environment of the relatively low- to middle-frequency (50 Hz to 200 kHz) bands as well as the existing high-frequency (200 kHz or more) range.

In addition, the contrast agent containing the iron oxide magnetic particles may be used and applied to various diagnostic imaging devices, and may be administered in a small amount to obtain sufficient images.

Furthermore, since the iron oxide magnetic particles have high structural stability due to the bond formed between iron oxide and the MXₙ compound, there is no concern about side effects that can be caused by each component, and they can be safely applied to the human body due to low toxicity.

The iron oxide magnetic particles according to one embodiment of the present invention may be used for the use of radiation therapy or thermotherapy for annihilating cancer cells.

Since the iron oxide magnetic particles according to the present invention have magnetism, they can be usefully used in diagnostic methods using magnetic properties.

According to one embodiment of the present invention, the present invention provides a cancer diagnosis method comprising the steps of (a) administering a composition containing the iron oxide magnetic particles to a patient suspected of cancer, and (b) detecting whether or not the magnetic particles inputted into the patient exist by using a magnetic resonance device. When the magnetic particles according to the present invention are administered, the contrast between the lesion and the normal tissue is clearly enhanced in, for example, MRI T1- and T2-weighted images, so that a visualized contrast effect can be confirmed. When the iron oxide magnetic particles of the present invention are administered, since cancer diagnosis can be performed without additional administration of a contrast agent, cancer diagnosis and treatment may be simultaneously performed with the iron oxide magnetic particles of the present invention.

When a cancer cell targeting material or a penetration force enhancing material is bonded to the iron oxide magnetic particles of the present invention, thermal diagnosis and treatment may be performed more efficiently under an external alternating magnetic field or radiation irradiation.

The iron oxide magnetic particles used in the contrast agent composition may be contained in an amount of 0.1 to 15% by weight, 1 to 15% by weight, 1 to 10% by weight, 3 to 10% by weight, or 4 to 8% by weight based on the total contrast agent composition.

The iron oxide magnetic particles are contained within the above-described range, and thus the iron oxide magnetic particles may not be accumulated in the body but may be discharged to the outside of the body, thereby significantly reducing toxicity as a contrast agent.

In one embodiment, the contrast agent may exhibit a contrast effect in a magnetic field having a frequency of 1 kHz to 1 MHz or less or an intensity of 20 Oe (1.6 kA/m) to 200 Oe (16 kA/m) or less. The alternating magnetic field irradiated after the contrast agent is administered to the subject may have a frequency of 1 kHz to 1 MHz or a frequency of 30 kHz to 120 KHz. In general, an alternating magnetic field of 1 MHz or more should be applied in order to convert a spin state from a singlet to a triplet, but in the present invention, the triplet transition is possible even under an alternating magnetic field of tens to hundreds of kHz. Also, the alternating magnetic field may have a magnetic field intensity of 20 Oe (1.6 kA/m) to 200 Oe (16.0 kA/m), 80 Oe (6.4 kA/m) to 160 Oe (12.7 kA/m), or 140 Oe (11.1 kA/m). The contrast agent according to one embodiment is useful in that it can be used even in an alternating magnetic field having a low magnetic field intensity and/or frequency that is harmless to the human body unlike conventional high-energy methods.

The contrast agent of the present invention has a feature that is not limited to devices that can be applied for image diagnosis. Since the contrast agent of the present invention has both a negative contrast agent component and a positive contrast agent component, it has high contrast to exhibit an excellent contrast effect. The iron oxide contrast agent of the present invention shows higher radiation absorption HU (hounsfield unit) value and CT contrast effect than conventional iodine-based (Iohexol or Iopamidol) or gold nano-CT contrast agents. Existing iodine-based contrast agents have been reported to show a value of 3000 HU (4.6 HU based on 1 mg) based on 647 mg/ml, and gold nanoparticles have been reported to show a value of about 5 to 50 HU based on 1 mg. Meanwhile, the iron oxide magnetic particles of the present invention shows a value of about 50 to 100 HU based on 1 mg.

In addition to the CT contrast effect, the present invention can be applied to X-ray imaging, magnetic resonance imaging (MRI), US, optical imaging, single photon emission computed tomography (SPECT), positron emission tomography (PET), magnetic particle imaging (MPI), flat imaging, and rigid, flexible or capsule endoscopy. Since the iron oxide magnetic particles according to the present invention can be used without limitation in various devices, for example, a patient can receive examinations of several devices at once after administering one dose of a contrast agent, and it may be possible to reduce unnecessary time for injecting other contrast agents according to conventional devices. For example, when a CT scan and an MRI scan are to be performed in the near future, as the CT contrast agent is mixed with the MRI contrast agent in the body of the subject, the test result may be unclear, and as the subject receives a different contrast agent for each test, the probability of causing toxicity increases. However, since the contrast agent of the present invention can be applied to various devices in a complex manner, such inconvenience can be reduced.

Another aspect provides a composition for diagnosing cancer including a contrast agent according to one embodiment.

The cancer may be gastric cancer, lung cancer, melanoma, uterine cancer, breast cancer, ovarian cancer, liver cancer, biliary tract cancer, gallbladder cancer, bronchial cancer, nasopharynx cancer, laryngeal cancer, pancreatic cancer, bladder cancer, colon cancer, rectal cancer, colorectal cancer, cervical cancer, brain cancer, bone cancer, skin cancer, blood cancer, kidney cancer, prostate cancer, thyroid cancer, parathyroid cancer, or ureter cancer.

The composition for diagnosing cancer may be administered to a subject in an oral or parenteral manner, and may include a pharmaceutically acceptable carrier so that it is suitable for each administration. Suitable pharmaceutically acceptable carriers and preparations are described in detail in Remington's work (Pharmaceutical Sciences 19th ed., 1995).

When the composition for diagnosing cancer is administered orally, it may be administered in solid preparations such as tablets, capsules, pills, and granules, or in liquid preparations such as solutions and suspensions.

When the composition for diagnosing cancer is administered parenterally, it may be administered by intravenous injection, subcutaneous injection, intramuscular injection, intraperitoneal injection, intralesional injection, intratumoral injection, or the like.

When the composition for diagnosing cancer is administered orally or parenterally as a liquid, it may be prepared as an aqueous solution or suspension using a commonly known solvent such as isotonic sodium chloride solution, Hank's solution, or Ringer's solution.

In one embodiment, the composition for diagnosing cancer may be for simultaneously treating cancer.

As mentioned above, the contrast agent of the present invention can annihilate cancer cells by thermotherapy. The term “thermotherapy” means exposing body tissues to a temperature higher than normal body temperature to annihilate lesion cells including cancer cells or to make these cells have higher sensitivities to radiation therapy or anticancer drugs. Cancer thermotherapy includes a systemic thermotherapy that increases the effect of cancer treatment in combination with radiation therapy/drug therapy and a local thermotherapy that annihilates cancer cells by injecting magnetic particles into target solid cancer and applying an external alternating magnetic field.

As such, since thermotherapy is capable of selectively annihilating cancer cells, it has an advantage of lowering side effects. However, existing thermotherapy technologies based on magnetic particles have problems in that the heating value of the particles themselves is low due to an external alternating magnetic field and the continuity thereof is limited so that the limitations of thermotherapy have been pointed out. The following two methods have conventionally been used in order to solve such problems:
(a) a method of increasing the intensity or frequency of an external alternating magnetic field in order to increase the heating phenomenon of the particles, or
(b) a method of increasing the concentration of particles to be injected into a living body.

However, (a) the method of increasing the intensity or frequency of the external alternating magnetic field may cause red spots around the skin and slight burns, wounds, inflammation, necrosis, etc. in regions with a lot of fat, may damage not only cancer tissues but also normal tissue cells, or may cause a result of lowering immunity. In addition, since such a method cannot avoid side effects due to harmfulness to the human body, it is prohibited to use it for pregnant women, patients with severe inflammation, patients with cardiac pacemakers, and patients with severe pleural effusion and ascites. As an alternative thereof, (b) the method of increasing the concentration of particles injected into a living body may increase the probability of accumulation of the particles in the body, and sometimes cause toxicity problems due to the chemical composition of the particle surface.

However, the iron oxide magnetic particles according to the present invention result in efficient heat generation when used for thermotherapy using an external alternating magnetic field or radiation equipment due to the effect of amplifying the internal quantum efficiency of iron oxide due to the difference in permittivity or electron capacitance by halogen groups. Accordingly, it may be possible to drastically lower the concentration of particles put into the living body compared to conventional iron oxide-based particles, and thereby also significantly improve bioaccumulation amount and toxicity problems. In conclusion, the present invention can dramatically overcome the disadvantages of the conventional technology, which were limited in use due to low heating value, despite the advantages of biocompatibility, chemical stability, and magnetic properties of iron oxide magnetic particles.

Hereinafter, Examples and the like will be described in detail to aid understanding of the present invention. However, embodiments according to the present invention can be modified in various different forms, and the scope of the present invention should not be construed as being limited to Examples below. The embodiments of the present invention are provided to more completely explain the present invention to those skilled in the art.

### EXAMPLES

### Examples 1 to 13: Synthesis of Iron Oxide Magnetic Particles containing MXₙ

### (a) Synthesis of Iron-Oleic Acid or Iron-Oleylamine Complex

To be the ratios of Tables 1 and 2 below, FeCl₃·6H₂O, sodium oleate (28 mmol) (Examples 1 to 4 and 9 to 13) or oleylamine (28 mmol) (Examples 5 to 8), 200 ml of hexane, 100 ml of ethanol, and 100 ml of deionized water were mixed and reacted at 110° C. for 6 hours with vigorous stirring. After cooling the reaction solution at room temperature, the transparent lower layer was removed using a separatory funnel, 100 ml of water was mixed with the brown upper organic layer and shaken, and then the lower water layer was removed again. This was repeated 3 times. The remaining brown organic layer was moved to a beaker and heated at 110° C. for 4 hours so that hexane was removed.

### (b) Synthesis of Iron Oxide Magnetic Particles Containing MXₙ

4.5 g (5 mmol) of the iron-oleic acid complex prepared in (a) above and 1.7 g (6 mmol) of oleic acid were mixed (Examples 1 to 4 and 9 to 13), or 4.208 g (5 mmol) of the iron-oleylamine complex prepared in (a) above and 1.6 g (6 mmol) of oleylamine were mixed (Examples 5 to 8). In addition, the types and contents of MXₙ in Tables 1 and 2 below were 7 ml of 1-eicosene and 13 ml of dibenzyl ether, respectively. The mixed solution was put into a round bottom flask and gas and water were removed in a vacuum state at 90° C. for about 30 minutes. Nitrogen was injected and the temperature was raised to 200° C. Thereafter, the temperature was raised to 310° C. at a rate of 3.3° C./min and then reacted for 60 minutes. After cooling the reaction solution, it was moved to a 50 ml conical tube, and 30 ml of ethanol and hexane were injected at a ratio of 1:1, and then centrifugation was performed to precipitate particles. After washing the precipitated particles with 10 ml of hexane and 5 ml of ethanol, the obtained precipitate was dispersed in toluene or hexane. Here, dibenzyl ether is decomposed into benzyl aldehyde and toluene at a temperature of 150° C. or higher, and participates in crystal formation by helping the formation of hydrogen bonds between iron oxo (—Fe—O—Fe—) and a transition metal element in which electrons are contained in the 5d orbital on the periodic table-halogen compound (MXₙ) by the radical generated from aldehyde. The size of the prepared particles was about 6 to 7 nm.

TABLE 1

| | | | Weight ratio of MXₙ | Weight ratio of MXn |
|---|---|---|---|---|
| | Iron complex | | based on iron | based on iron oxide |
| | (iron-oleic acid or | | complex (input | (analysis value after |
| | iron oleylamine | MXₙ type | amount during | material formation: |
| Remark | content g) | (content g) | preparation) | based on ICP) |
| Example 1 | 4.501 g | HfI₄ 0.023 g | 0.005 | 0.0045 |
| | (5 mmol) | (0.033 mmol) | | |
| Example 2 | 4.501 g | HfI₄ 0.135 g | 0.030 | 0.028 |
| | (5 mmol) | (0.197 mmol) | | |
| Example 3 | 4.501 g | HfI₄ 0.270 g | 0.060 | 0.058 |
| | (5 mmol) | (0.393 mmol) | | |
| Example 4 | 4.501 g | HfI₄ 0.45 g | 0.100 | 0.093 |
| | (5 mmol) | (0.763 mmol) | | |
| Example 5 | 4.208 g | HfI₄ 0.021 g | 0.005 | 0.0047 |
| | (5 mmol) | (0.031 mmol) | | |
| Example 6 | 4.208 g | HfI₄ 0.126 g | 0.030 | 0.028 |
| | (5 mmol) | (0.184 mmol) | | |
| Example 7 | 4.208 g | HfI₄ 0.214 g | 0.060 | 0.057 |
| | (5 mmol) | (0.312 mmol) | | |
| Example 8 | 4.208 g | HfI₄ 0.421 g | 0.100 | 0.098 |
| | (5 mmol) | (0.614 mmol) | | |

TABLE 2

| | | | Weight ratio of MXₙ | Weight ratio of MXn |
|---|---|---|---|---|
| | | | based on iron | based on iron oxide |
| | Iron complex | | complex (input | (analysis value after |
| | (iron-oleic acid | MXₙ type | amount during | material formation: |
| Remark | content g) | (content g) | preparation) | based on ICP) |
| Example 9 | 4.501 g | HfF₄ 0.270 g | 0.060 | 0.059 |
| | (5 mmol) | (1.061 mmol) | | |
| Example 10 | 4.501 g | HfBr₄ 0.270 g | 0.060 | 0.057 |
| | (5 mmol) | (0.542 mmol) | | |
| Example 11 | 4.501 g | HfCl₄ 0.270 g | 0.060 | 0.058 |
| | (5 mmol) | (0.843 mmol) | | |
| Example 12 | 4.501 g | HfI₄ 0.270 g | HfI₄: 0.060 | HfI₄: 0.058 |
| | (5 mmol) | (0.393 mmol) | | |
| | | CuI 0.270 g | CuI: 0.060 | CuI: 0.057 |
| | | (1.416 mmol) | | |
| Example 13 | 4.501 g | HfI₄ 0.135 g | HfI₄: 0.030 | HfI₄: 0.028 |
| | (5 mmol) | (0.197 mmol) | | |
| | | CuI 0.270 g | CuI: 0.060 | CuI: 0.057 |
| | | (1.461 mmol) | | |
| Com. Ex. 1 | 4.501 g | CuI 0.270 g | 0.060 | 0.057 |
| | (5 mmol) | (1.416 mmol) | | |
| Com. Ex. 2 | 4.501 g | CuBr 0.270 g | 0.060 | 0.058 |
| | (5 mmol) | (1.882 mmol) | | |

(c) Preparation of Iron Oxide Magnetic Particles Coated with Hydrophilic Ligand (Polyacrylic Acid)

While 2 g of polyacrylic acid and 40 ml of tetraethylene glycol were heated at 110° C., 150 mg of the iron oxide magnetic particles containing MXₙ of Examples and Comparative Examples above dispersed in 5 ml of hexane were injected with a syringe. It was stirred and reacted at 280° C. for 8 hours. After cooling the reaction solution, 20 ml of 0.01 N HCl was put thereinto and particles attracted to the magnet were collected. After repeating this twice, a precipitate was obtained using ethanol and finally dispersed in water.

### Experimental Example: XPS and TEM Analysis

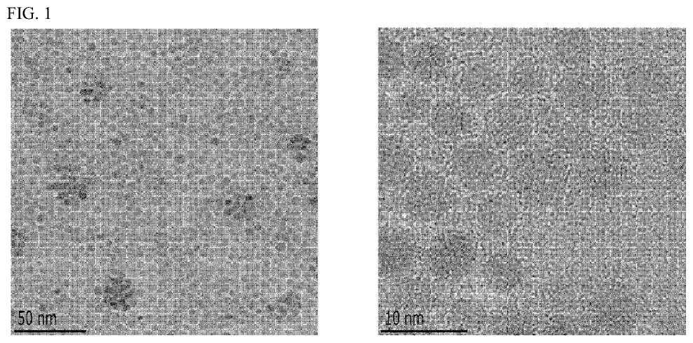

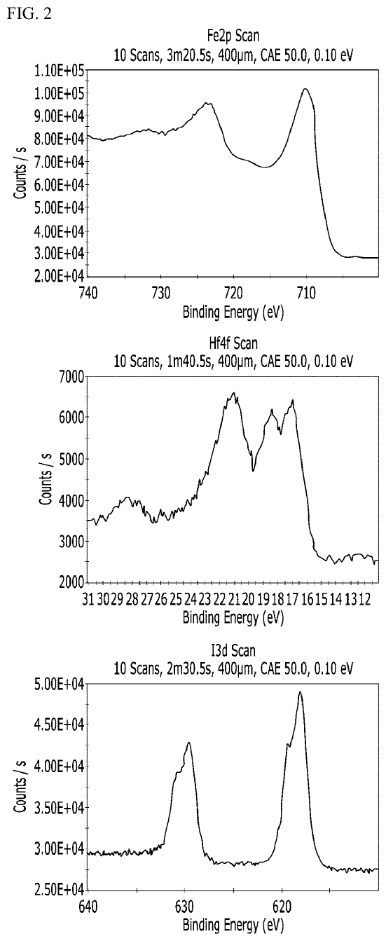

The TEM photographs of iron oxide magnetic particles of Example 3 above are shown in FIG. 1. In addition, the results of XPS component analysis of the iron oxide magnetic particles of Example 3 above are shown in FIG. 2.

### Experimental Example: Analysis of Temperature Change According to the Weight Ratio of Iron Oxide and MXₙ under an External Alternating Magnetic Field

After coating the particles prepared in Examples and Comparative Examples above with polyacrylic acid, which is a hydrophilic ligand, self-induction heating ability was tested. After diluting each of Examples and Comparative Examples in deionized water to a concentration of 20 mg/ml, an alternating magnetic field was applied, and the temperature changes were measured using a thermocouple (OSENSA, Canada) (alternating current frequency and magnetic field intensity used: f=108.7 kHz, H=11.4 kA/m). The results are shown in Table 3 below.

A system of performing heating by inducing an alternating magnetic field consists of four main subsystems of (a) a variable frequency and amplitude sine wave function generator (20 MHz Vp-p, TG2000, Aim TTi, USA), (b) a power amplifier (1,200 Watt DC Power Supply, QPX1200SP, Aim TTi, USA), (c) an induction coil (number of revolutions: 17, diameter: 50 mm, height: 180 mm) and a magnetic field generator (Magnetherm RC, nanoTherics, UK), and (d) a temperature change thermocouple (OSENSA, Canada).

TABLE 3

| | Temperature reached per unit time |
|---|---|
| | (Standard: 1 minute) |
| | Start measurement at 25° C. |
| Example 1 | 32 |
| Example 2 | 55 |
| Example 3 | 80 |
| Example 4 | 30 |
| Example 5 | 28 |
| Example 6 | 51 |
| Example 7 | 69 |
| Example 8 | 27 |
| Example 9 | 53 |
| Example 10 | 48 |
| Example 11 | 43 |
| Example 12 | 78 |
| Example 13 | 84 |
| Comparative Example 1 | 86 |
| Comparative Example 2 | 57 |

### Experimental Example: Measurement of Specific Loss Power (SLP)

Since the heating value of particles varies depending on the physical and chemical properties and the intensity and frequency of an external alternating magnetic field, most research results show the heating ability of particles as SLP and ILP. SLP is the electromagnetic force lost per unit of mass, and expressed in Watts (W) per kg. Since the conditions of f (frequency) and H (magnetic field intensity) may be different for each experiment, the thermotherapy effects between the particles can be compared by converting the SLP value into an ILP value using the equation [ILP=SLP/(f H²)].

For the SLP measurement, an alternating magnetic field generator (Magnetherm RC, Nanotherics) of a series resonant circuit controlled by a pick-up coil and an oscilloscope was used. The measurement was performed under adiabatic conditions of f=108.7 kHz and H=11.4 kA/m, and the temperatures were measured using an optical fiber IR probe.

SLP values were measured by adjusting the particles of Examples and Comparative Examples to a concentration of 20 mg/ml. The results are shown in Table 4 below.

TABLE 4

| | SLP value |
|---|---|
| Example 1 | 1.26 |
| Example 2 | 5.27 |
| Example 3 | 9.50 |
| Example 4 | 1.17 |
| Example 5 | 1.09 |
| Example 6 | 4.82 |
| Example 7 | 7.24 |
| Example 8 | 1.05 |
| Example 9 | 5.08 |
| Example 10 | 4.22 |
| Example 11 | 3.78 |
| Example 12 | 8.98 |
| Example 13 | 9.36 |
| Comparative Example 1 | 9.81 |
| Comparative Example 2 | 5.14 |
| Control group | |

### Experimental Example: In Vivo Cancer Treatment Effect Checking Test

It was confirmed that cell annihilation by thermotherapy using the particles of Examples and Comparative Examples of the present invention effectively occurred even in vivo. After Panc-1 cells were transplanted into Balb/c nude mice, when the size of the cancer tissue was about 100 mm³, 150 μl of an aqueous solution obtained by dispersing the compositions containing Examples and Comparative Examples in deionized water was subcutaneously administered, and then an alternating magnetic field generator (100 kHz, 80 G) was applied for 30 minutes to perform thermotherapy and check the volume of the cancer for 28 days. In the case of the control group below, the tumor sizes was measured when no separate treatment was performed.

The results are shown in Table 5 below.

TABLE 5

| | Final tumor size after treatment (mm³) | | | | |
|---|---|---|---|---|---|
| | (Specific week) | | | | |
| Remark | (Week 0) | (Week 1) | (Week 2) | (Week 3) | (Week 4) |
| Example 1 | 101 | 145 | 182 | 205 | 370 |
| Example 2 | 99 | 78 | 58 | 55 | 46 |
| Example 3 | 100 | 67 | 30 | 28 | 22 |
| Example 4 | 103 | 150 | 187 | 211 | 390 |
| Example 5 | 97 | 160 | 199 | 230 | 411 |
| Example 6 | 102 | 85 | 67 | 60 | 52 |
| Example 7 | 98 | 80 | 61 | 58 | 50 |
| Example 8 | 101 | 162 | 203 | 241 | 421 |
| Example 9 | 98 | 82 | 61 | 57 | 48 |
| Example 10 | 100 | 91 | 75 | 63 | 58 |
| Example 11 | 103 | 101 | 85 | 71 | 66 |
| Example 12 | 97 | 69 | 35 | 32 | 28 |
| Example 13 | 99 | 67 | 32 | 30 | 25 |
| Comparative | 98 | 65 | 21 | 18 | 15 |
| Example 1 | | | | | |
| Comparative | 101 | 74 | 51 | 31 | 24 |
| Example 2 | | | | | |
| Control | 97 | 171 | 290 | 440 | 730 |
| group | | | | | |

### Experimental Example: Radiation Absorption Coefficient (HU) Measurement

Radiation (X-ray) absorption coefficients (HU) were measured using the particles of Examples, Comparative Examples and Control group above, and the results are shown in Table 6 below. At this time, Bruker's Skyscan 1172 Micro CT was used as a measuring device, and the radiation absorption coefficients (HU) were calculated as follows.

### CT Hounsfield Unit (HU, X-ray Absorption Coefficient)

$$HU = 1000 \times \frac{μ - {μ}_{water}}{{μ}_{water} - {μ}_{air}}$$

(μ: Relative linear attenuation coefficient)

TABLE 6

| | Radiation absorption coefficient (HU) |
|---|---|
| Remark | per unit weight (based on 1 mg) |
| Example 1 | 58 |
| Example 2 | 78 |
| Example 3 | 99 |
| Example 4 | 48 |
| Example 5 | 46 |
| Example 6 | 69 |
| Example 7 | 88 |
| Example 8 | 45 |
| Example 9 | 73 |
| Example 10 | 66 |
| Example 11 | 62 |
| Example 12 | 96 |
| Example 13 | 97 |
| Comparative Example 1 | 97 |
| Comparative Example 2 | 18 |
| Control group 1 (iron oxide | About 10 |
| Fe₃O₄, see reference: Sci | |
| Rep., 8, 12706 (2018)) | |

### Experimental Example: Thermogravimetric Analysis (TGA) of Iron Oxide Magnetic Particles

In order to examine the thermal stability of the iron oxide magnetic particles of the present invention (purpose is to confirm how stably the halogen elements are bound in the iron oxide magnetic particles), thermogravimetric analysis (TGA) was performed by using Scinco's S-1000. Specifically, the particles of Examples, Comparative Examples and Control group were subjected to TGA measurement up to 200° C. at a rate of 20/min under nitrogen to perform comparison. The results are shown in Table 7.

TABLE 7

| Remark | Weight reduction rate (%) |
|---|---|
| Example 1 | 5.8 |
| Example 2 | 3.5 |
| Example 3 | 2.0 |
| Example 4 | 6.1 |
| Example 5 | 5.9 |
| Example 6 | 3.8 |
| Example 7 | 2.7 |
| Example 8 | 6.2 |
| Example 9 | 3.6 |
| Example 10 | 4.1 |
| Example 11 | 4.5 |
| Example 12 | 2.1 |
| Example 13 | 1.8 |
| Comparative Example 1 | 3.0 |
| Comparative Example 2 | 6.0 |
| Control group 1 (iron oxide Fe₃O₄) | 15 |
| Control group 2 (doped with 6% by | 17 |
| weight of iron oxide-KI) | |
| Control group 3 (doped with 6% by | 13 |
| weight of iron oxide-MgI₂) | |

### Experimental Example: Microwave Stability Analysis of Iron Oxide Magnetic Particles

The particles of the Examples, Comparative Examples, and Control groups were irradiated for 15 minutes each at conditions of 2,400 to 2,500 MHz and 1000 W using a microwave device made in the US by CEM. After microwave irradiation, the contents of halogen elements were measured in a prodigy High Dispersion ICP measuring instrument equipped with a halogen option from A Teledyne Leeman Labs to confirm whether the particles were decayed or not. The results are shown in Table 8.

TABLE 8

| | Measured halogen elements |
|---|---|
| Remark | (ppm) |
| Example 1 | 19 |
| Example 2 | 10 |
| Example 3 | 3 |
| Example 4 | 22 |
| Example 5 | 21 |
| Example 6 | 13 |
| Example 7 | 8 |
| Example 8 | 23 |
| Example 9 | 15 |
| Example 10 | 18 |
| Example 11 | 4 |
| Example 12 | 3 |
| Example 13 | 2 |
| Comparative Example 1 | 25 |
| Comparative Example 2 | 30 |
| Control group 1 (iron oxide Fe₃O₄) | 0 |
| Control group 2 (doped with 6% by | 65 |
| weight of iron oxide-KI) | |
| Control group 3 (doped with 6% by | 57 |
| weight of iron oxide-MgI₂) | |

1. Iron oxide magnetic particles containing iron oxide and MXₙ , wherein M as a transition metal containing electrons in a 5d orbital on the periodic table includes one or more selected from the group consisting of Hf, Ta, W, Re, Os, Ir, Pt, Au, and Hg, X includes one or more selected from the group consisting of F, Cl, Br, and I, and n is an integer of 1 to 6.

2. The iron oxide magnetic particles of claim 1, wherein X includes a radioactive isotope of X or mixtures of radioactive isotopes of X.

3. The iron oxide magnetic particles of claim 1, wherein at least a portion of the surface of the iron oxide magnetic particles is additionally coated with a hydrophilic polymer to form a complex.

4. The iron oxide magnetic particles of claim 1, wherein the iron oxide is derived from complexes of iron and one or more compounds selected from the group consisting of an aliphatic carboxylic acid salt having 4 to 25 carbon atoms and an amine-based compound.

5. The iron oxide magnetic particles of claim 1, wherein the iron oxide includes one or more selected from the group consisting of Fe₁₃O₁₉, Fe₃O₄ (magnetite), γ-Fe₂O₃ (maghemite), α-₂O₃ (hematite), β-Fe₂O₃ (beta phase), ε- Fe₂O₃ (epsilon phase), FeO (wustite), FeO₂ (iron dioxide), Fe₂O₅, Fe₅O₆, Fe₅O₇, Fe₂₅O₃₂, ferrite type, and delafossite.

6. The iron oxide magnetic particles of claim 3, wherein the hydrophilic polymer includes one or more selected from the group consisting of polyethylene glycol, polyethylene amine, polyethyleneimine, polyacrylic acid, polymaleic anhydride, polyvinyl alcohol, polyvinylpyrrolidone, polyvinyl amine, polyacrylamide, polyethylene glycol, phosphoric acid-polyethylene glycol, polybutylene terephthalate, polylactic acid, polytrimethylene carbonate, polydioxanone, polypropylene oxide, polyhydroxyethyl methacrylate, starch, dextran derivatives, amino sulfonic acids, sulfonic acid peptides, silica, and polypeptides.

7. The iron oxide magnetic particles of claim 1, wherein the iron oxide magnetic particles contain MXₙ at a weight ratio of 1:0.005 to 0.08 based on iron oxide.

8. The iron oxide magnetic particles of claim 1, wherein the particles have a diameter of 0.1 nm to 1,000 nm.

9. The iron oxide magnetic particles of claim 1, wherein the iron oxide magnetic particles are used at a frequency of 1 kHz to 1 MHz.

10. The iron oxide magnetic particles of claim 1, wherein the iron oxide magnetic particles are used in a magnetic field having an intensity of 20 Oe (1.6 kA/m) to 200 Oe (16.0 kA/m).

11. The iron oxide magnetic particles of claim 1, wherein the iron oxide magnetic particles are used in radiation.
